SEQUENCE LISTING (9 sequences)

SEQ ID NO: 1 - DNA, 54 bp, Artificial Sequence
  5′-phosphorylated synthetic oligodeoxyribonucleotide
gggttaccaccttcattggaaaacgttcttcggggcgttcttaggtggtaaccc

SEQ ID NO: 2 - DNA, 29 bp, Artificial Sequence
  5′-phosphorylated synthetic oligodeoxyribonucleotide
tcattggaaacgttcttcggggcgttctt

SEQ ID NO: 3 - DNA, 58 bp, Artificial Sequence
  5′-phosphorylated synthetic oligodeoxyribonucleotide
cctaggggttaccaccttcattggaaaacgttcttcggggcgttcttaggtggtaacc

SEQ ID NO: 4 - DNA, 62 bp, Artificial Sequence
  5′-phosphorylated synthetic oligodeoxyribonucleotide
ccctaggggttaccaccttcattggaaaacgttcttcggggcgttctttccccaatggtgga

SEQ ID NO: 5 - DNA, 62 bp, Artificial Sequence
  5′-phosphorylated synthetic oligodeoxyribonucleotide
cccttccaccattggggatcattggaaaacgttcttcggggcgttcttaggtggtaacccct

SEQ ID NO: 6 - DNA, 54 bp, Artificial Sequence
  5′-phosphorylated synthetic oligodeoxyribonucleotide
aggggttaccaccttcattggaaaacgttcttcggggcgttcttaggtggtaac

SEQ ID NO: 7 - DNA, 58 bp, Artificial Sequence
  5′-phosphorylated synthetic oligodeoxyribonucleotide
ctaggggttaccacctacaaaaaaaaacgaaattcggggcgaagggaggtggtaaccc

SEQ ID NO: 8 - DNA, 58 bp, Artificial Sequence
  5′-phosphorylated synthetic oligodeoxyribonucleotide
ctaggggttaccaccttctatagaaaacgttcttcggggcgttcttcatcggtaaccc

SEQ ID NO: 9 - DNA, 58 bp, Artificial Sequence
  5′-phosphorylated synthetic oligodeoxyribonucleotide
agctgggttaccaccttcattggaaaacgttcttcggggcgttcttaggtggtaaccc

(19) United States
(12) Patent Application Publication
(10) Pub. No.: US 2012/0093804 A1
(43) Pub. Date: Apr. 19, 2012

(54) Concatamers for Immunemodulation

(75) Inventors: Matthias Schroff, Berlin (DE); Burghardt Wittig, Berlin (DE); Manuel Schmidt, Berlin (DE); Christiane Kleuss, Berlin (DE)

(73) Assignee: Mologen AG, Berlin (DE)

(21) Appl. No.: 13/266,905

(22) PCT Filed: Apr. 30, 2010
(86) PCT No.: PCT/EP10/55903

§ 371 Date: Jan. 9, 2012

(30) Foreign Application Priority Data
Apr. 30, 2009 (EP) 09075220.5

(51) Int. Cl. A61K 39/00; A61P 37/04; A61K 47/26; C07H 21/04; A61K 39/395

(52) U.S. Cl. 424/130.1; 536/23.1; 424/184.1; 424/278.1

(57) ABSTRACT

The invention relates to a polymeric, non-coding nucleic acid molecule for modulation of the activity of the human and animal immune system as well as a method for the manufacture thereof and a vaccine, comprising the polymeric, non-coding nucleic acid molecule, wherein polymeric, non-coding nucleic acid molecules may be understood as non-coding nucleic acid molecules, comprising at least four covalently bound molecules (tetramer) or are assemblies of more non-coding nucleic acid molecules (high molecular polymers) which are covalently bound to each other.

The invention relates to a polymeric, non-coding nucleic acid molecule for the modulation of the activity of the human and animal immune system as well as a method for the manufacture thereof and a vaccine, comprising the polymeric, non-coding nucleic acid molecule, wherein polymeric, non-coding nucleic acid molecules may be understood as non-coding nucleic acid molecules, comprising at least four covalently bound molecules (tetramer) of said non-coding nucleic acid molecules.

As the adaptive immune response starts with a delay (3-5 days) after selection of the specific lymphocytes for the respective pathogen, their clonal expansion and differentiation to effector cells, but then provides a long lasting protection for the respective pathogen by forming an immunological memory, the cells of the innate immune system recognize pathogens via conserved pathogen associated molecular patterns (PAMP) by germ cell encoded receptors and react immediately. Different reactions belong to different kinds of cell types like the secretion of cytokines (e.g. IL-1, IL-6, TNF-α) and chemokines (e.g. IL-8/CXCL8, MIP-1α/β, MCP-1), the activation of effectors mechanisms (phagocytosis, respiratory discharge, liberation of bactericide or cytotoxic substances or lytic granules), the expression of co-stimulatory molecules (CD80, CD86) as well as the enhanced expression of MHC-molecules. Thereby on one hand effector cells are recruited and activated, which are able to eliminate the entered pathogen, on the other hand the cells of the adaptive immune system receive the necessary signals for their activation.

In order to improve the immune response CpG-oligonucleotides (CpC-ODN) have been used as a new class of immune modulating molecules. Such non-methylated CG-motives can be found in bacterial DNA and represent a “danger signal” for the immune system. As pathogen associated molecular pattern (PAMP) they cause the unspecific activation of the innate immune system (Krieg, Nat. Med 2003, 9: 831-835). CpG-ODN induce via the cytokines interleukine-12, interferon-γ and tumor necrosis factor-α a T_{H}1-based immune response.

Immune stimulatory nucleic acids (ISS), comprising said CpG-ODN, have a length of several bases and comprise no open reading frame for the expression of proteins.

The ISS represent linear nucleic acid molecules, which ends are open (free hydroxyl- and phosphate groups) or protected by synthetic groups.

The stimulation of the cellular immune response allows influencing the feedback loops, which will not result in a satisfactory immune activity for the patient without intervention.

The modification of CpC-ODN with a phosphothioate-backbone, which is used for stabilizing the CpG-DNA, has several severe disadvantages. The noted toxicity belongs especially to this (Heikenwalder 2004, Levin 1999) as well as unspecific binding to proteins (Brown 1994).

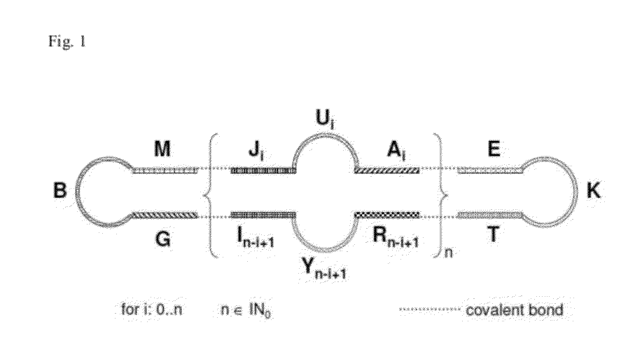

Due to this a new class of covalently closed immunemodulatory DNA was developed (WO 01/07055/EP 1196178). These DNA-molecules consist of two chemically synthesized DNA-ODN, with a self complementary part at the 5′- and at the 3′-end with palindromic, overlapping ends, so that ligation of both DNA-molecules results in a covalently closed molecule. These DNA-molecules with CG-motives in the non-complementary part show a similar activity as CpG-ODN (enhanced expression of the surface molecules CD80, CD40, MHC on B-cells and secretion of IL-6, IFN-γ, IFN-α IL-12, TNFα by PBMC), but they show in comparison to CpG-ODN with phosphorothioate backbone differences with regard to the expression pattern of the induced cytokines and a clearly lesser toxicity in mice. This immunemodulatory DNA from the state of the art has with regard to the modulation of the activity of the human and animal immune system several disadvantages. It is not possible, to modulate the activity of the human and animal immune system in a desired degree, especially to activate it. The molecules according to WO 01/07055, as shown for example in FIG. 1 or in claim 11, consist of several deoxyribonucleotide rests which form a partly single stranded dumbbell-shaped and covalently closed DNA molecule, which is designated within the scope of the present invention as a dimer. According to WO 01/07055 the monomeric nucleic acids used as starting material were heated before ligation, receiving uniform molecules of nucleic acids, each consisting of a dumbbell-shaped dimer (compare FIG. 1 of the WO 01/07055). The resulting nucleic acid is a dumbbell according to FIG. 1 of WO 01/07055. Monomer in the sense of the invention does not designate a structure consisting for instance out of a single base, but designates a nucleic acid, which consists itself out of several deoxyribonucleotide mono-phosphates (compare FIG. 1 or claim 11 of WO 01/07055) which form due to their defined base sequence or a defined three-dimensional conformation jointly the monomer-typical properties.

Coming from this state of the art it is an objective of the present invention to provide suitable immunemodulatory DNA molecules, which initiate an improved immune response, as well as a method for their manufacture as well as vaccines, comprising said immunemodulatory DNA-molecules.

Immune modulation means in the context of the present invention that the mediator and effector cells of the immune system, thus mainly the presently known thymocytes with helper function and the cytotoxic thymocytes, B-cells and so called NK (natural killer)-cells, macrophages and monocytes as well as dendritic cells and their precursors, as well as cell populations with so far not clearly identified functions which have a function within the immune system, are stimulated by the use of nucleic acid molecules for proliferation, migration, differentiation or their activity Immune modulation means, that besides a general improvement of the immune response in the above defined sense also the type or character of the immune reaction will be influenced, whether by affecting a beginning or maturing immune reaction or by changing an established reaction with regard to their character.

The molecule with improved immunemodulatory properties claimed by the present invention is compared with the dimeric substances from the WO 01/07055 a polymeric, non-coding nucleic acid molecule. A polymeric nucleic acid molecule should be understood as so-called high molecular concatemer. The invented polymeric molecule can be manufactured by a method, comprising the following steps:
providing a 5′-phosphorylated deoxyribonucleotide acid,
alcohol precipitation and subsequent drying of the precipitate at 50° C. or lyophilisation of the DNA molecule at 50° C., especially in the presence of MgC1₂, until a dry residue is obtained, followed by resuspension in a buffer.
adding T4-DNA-ligase, thereby producing a reaction mixture, and
incubation of the reaction mixture at 37° C. for at least 30 minutes.

Concatemers comprise covalently bound monomer units, which are in their entirety circularly closed, having within the distance of the constitutive monomers double stranded parts and immunemodulatory CG-motives preferably in the single stranded parts. It was completely surprising, that these polymers comprising tetramers, hexamers or high molecular assemblies of covalently closed immunemodulartory DNA have a surprisingly improved effect in comparison to dimeric molecules from the state of the art.

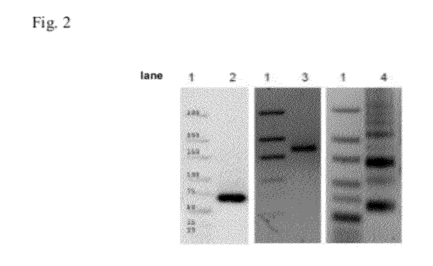

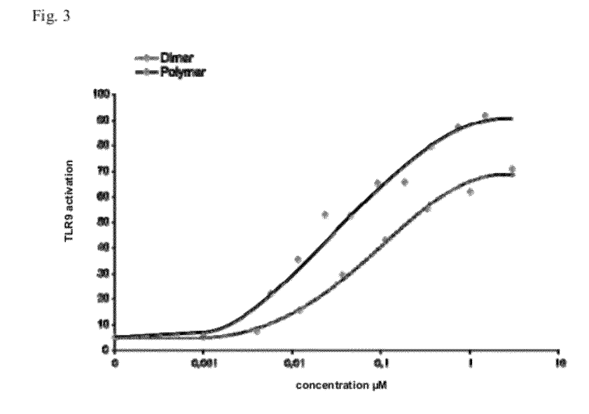

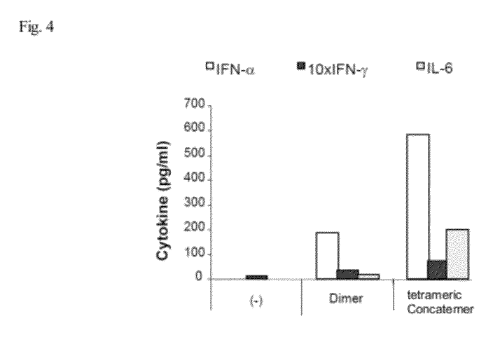

The claimed polymeric molecules are shown in FIGS. 1 and 2 with regard to the molecular properties and in FIGS. 3 and 4 with regard to the functional properties, which result from the application of the method for the manufacture for a person skilled in the art. The use of nucleic acid molecules with palindromic 5′-respectively 3′-ends as educts in the described method results in polymers with different sizes, from which only the claimed tetramers or high molecular assemblies fulfill the highly potent function. Since the characterization by structural features is not feasible due to the extended and diverse molecule size, the characterization of the polymers via their method of manufacture is very precise. The new method provides a different product than the one described in the state of the art. This can be demonstrated by clear differences in the properties of the dimers and polymers according to the invention as shown in FIG. 3. The high molecular polymers according to the invention are surprisingly better suited for immune modulation then the non-polymeric structures known from the state of the art.

The molecules according to the invention can also be manufactured by providing 5′-phosphorylated deoxyribonucleotide acids in water, if they are purified with an equivalent method to a polyacrylamide gel electrophoresis, especially by the combined purification with

HPLC followed by FPLC. It is known by a person skilled in the art, that by the combination of several high performance methods like HPLC or FPLC an analogue grade of purification to a PAGE-purification can be obtained.

Surprisingly the chronology of the single steps of the method results in a mulitmeric molecule, comprising circular stably and covalently with each other bound monomers with at least 24 nucleotides. The simultaneously formed high molecular polymers comprise always a even number of monomeric components. The formed chain of molecules does not comprise free 5′- or 3′ ends. The monomers forming via intermolecular esterification the molecule according to the invention are characterized by:
comprising a part of at least 2 sequential nucleotides, which forms under suitable conditions with another part of the monomer a double stranded stem,
between these reverse complementary parts are at least 4 nucleotides
CG-motives, which are recognized by cellular structures, are preferably present in the single stranded part,
modified nucleotides can also be part of a single stranded area, which are covalently linked to fatty acids, sugars or amino acids.

A molecule according to the invention comprises at least four monomers and is formed with regard to its conformation during the above-mentioned synthesis. The monomers are forming via intermolecular bonds to catena of two, four, six or more by formation of covalent bond. This results in the formation of so called di-, tetra- or hexamers, which are all designated as polymers with exception of the dimers.

A molecule according to the invention can be also defined as concatemer. In a preferred embodiment it is intended, that the molecule according to the invention is a concatermeric molecule, wherein at least four loops of individual monomers are linked with each other, preferably linear so that preferably two especially preferred several double-stranded parts are each separated from each other by single stranded loop elements.

A molecule according to the invention is able to modulate the activity of the human or animal immune system better compared to molecules of the state of the art. The molecules from the state of the art are the known immune modulatory nucleic acid sequences, which are effective as low molecular dumbbell-shaped structures. The most known, immune modifying short oligodeoxyribonucleotide acids comprise an unmethylated cytosine-guanosine-motive. A physiological effect of such nucleic acids is also understood as immune modulation respectively modulation of the activity of the immune system within the sense of the invention. The EP 1 196 178 discloses additionally several molecules, consisting of a stem with at least one loop, as they are disclosed for example in the FIGS. 1 and 2 of EP 1 196 178. Within the sense of the present invention such molecules are dimeric structures. The present invention does not comprise such dimers. It has to be noted that the term polymer is used with several different meanings in science. A polymer may be for instance a longer nucleic acid as well as a structure comprising several of the same or similar molecules formed to a larger assembly. An polymer within the sense of the invention designates catena of molecules, comprising at least four monomers. If the preferred monomers are used, the molecular weight of the resulting tetramer corresponds to about 170 kDa (comp. FIG. 2). Polymers within the sense of the invention would be for instance several stem-loop-structures as depicted in FIG. 1, assembling with several of the same or similar stem-loop-structures to a higher structure (a polymer). As polymer are all molecules according to the invention designated which are larger than 23 kDa. The described conditions for the reaction cause during the ligation a transient attachment of the monomers, which can be esterified by ligase. A resulting polymer will be formed during the synthesis with respect to its confirmation only under the special reaction conditions. It is not possible to manufacture the high molecular polymers from dimers that have already been formed. The monomer structures forming the polymer are covalently linked to each other. A formed polymer is stabile with respect to heat or denaturing agents, which means vice versa that the dimers can not be obtained with simple physical means out of a high molecular molecule according to the invention.

It is surprising, that comparatively simple method steps can obtain such polymeric structures having improved and not obvious properties compared to dimeric structures. The production of high molecular assemblies for instance can be performed via centrifugation, gel electrophoresis or column chromatography to detect and obtain high complex structures, like for instance tetramers, hexamers or others, which have compared to dimers improved properties with regard to the modulation of the immune system (compare FIGS. 3 and 4). Different forms of immune modulation in lab organisms or humans prove this.

All deoxyribonucleic acids according to the following characterization can be used in the polymerization method.

5′-P—W—S-3′, wherein
P, W, S are nucleic acids, linked to each other in the listed reading order via phosphodiester links “—”,
the sequence of the nucleic acid P, W or S comprises at least one motive of the deoxyribonucleotide sequence CG.
W is at least 4 nucleotides long and
the sequences of the nucleic acid parts S and P are reverse complementary to each other.

The resulting polymers are in compliance with the formula:

W—S—{P—W—S}ₙ—P—W—S—{P—W—S}ₙ—P ∀ n ε IN₀, wherein
the nucleic acid P at the right side in the formula is covalently linked to the left nucleic acid W
“n” describes the degree of contcatemerization by indicating the number of inner monomer units.

Because the claimed polymers receive their properties by their confirmation, a polymeric structure within the sense of this application can be assembled from non-sequence identical monomers. The nucleic acid group W may comprise in this connection molecules with the sequences of B, U, K, Y, the nucleic acid group P may comprise molecules with the sequences of J, E, R, G and the nucleic acid group S may comprise sequences of M, A, T, I. Depending on the multitude n of monomers in the core part of the resulting polymer, several different sequence parts J-U-A respectively R—Y—I are present, which do not have to be sequence identical with regard to each other what is shown by index “i” respectively “n−i+1”. The claimed polymers with sequence identical or sequence different monomer components conform to the formula:

B-M-{[Jᵢ-Uᵢ-Aᵢ]_{0 . . . n}}ₙ-E-K-T-{[Rₙ₋ᵢ₊₁-Yₙ₋ᵢ₊₁1ₙ₋ᵢ₋₁]_{0 . . . n}}ₙ-G ∀ n ε IN₀, wherein
A, B, E, G, I, J, K, M, R, T, U, Y are deoxyribonucleotide molecules and
the sequence of component i of a nucleic acid molecule may be different, but does not have to, compared to the (i+1) of the same molecule and
at least one nucleic acid comprises a motive with the deoxyribonucleotide sequence CG and
B, Uᵢ, K and Yₙ₋ᵢ₊₁ are predominantly single stranded and
B, Uᵢ, K and Y ₙ₋ᵢ₊₁ are each assembled of at least 4 deoxyribonucleotides and
the sequences of Jᵢ to 1ₙ₋ᵢ₊₁, Aᵢ to Rₙ₋ᵢ₊₁ M to G respectively E to T are reveres complementary to each other and
G is covalently linked via a phosphodiester bound to B.

Preferably a polymer according to the invention is characterized in that the deoxyribonucleic acid used in the method comprises the following sequence:

(SEQ ID No. 1)
5′-GGGTTACCACCTTCTATAGAAAACGTTCTTCGGGGCGTTCTTC-
ATCGGTAACCC-3′

wherein the deoxyribonucleic acid has a length from 20 to 400 nucleotides.

The synthesis of the educts with the preferred sequences results in molecules, which are surprisingly suitable for the modulation of the immune response. It is especially preferred if the base sequence within the single stranded molecule parts is partly or completely in accordance with the sequence

(SEQ ID No. 2)
5′-TCATTGGAAACGTTCTTCGGGGCGTTCTT-3′

It is surprising that the presence of these sequences results in a very good activity of the concatameric polymers. Within the concatameric structure of a molecule the partly single stranded covalently closed chains of deoxyribonucleotides are responsible for the long term effect of the molecules in target organism in which they are introduced.

In a further preferred embodiment of the invention it is intended, that the monomer comprises the base sequence N¹N²CGN³N⁴, wherein N¹N² is an element of the group of GT, GG, GA, AT or AA, N³N⁴ is an element of the group CT or TT, as well as C deoxycytosine, G deoxyguanosine, A deoxyadenosine and T deoxythymidine.

In an especially preferred embodiment it is intended, that the base sequence N¹N²CGN³N⁴ is positioned within the single stranded part of the closed chain of deoxyribonucleotides. Especially these preferred molecules show very strong effects during modulation of the immune systems.

It is a matter of course, that a molecule according to the invention may have one or more substitutes bound via covalent bonds. Such substitutes may be e.g. peptides, proteins, saccharides, lipids, antigenic structures, DNA and/or RNA.

The invention relates besides the above mentioned structural and functional features of the product also to a method for the manufacture of the molecule comprising the following steps:
providing a 5′-phosphorylated DNA molecule in water purified by polyacrylamide gel electrophoresis,
lyophilisation at 50° C. until a dry residue is received and subsequent resuspension in a buffer,
adding a T4-DNA-ligase, forming a reaction mixture and
incubation of the reaction mixture at 37° C. for at least 30 minutes, or
providing a deoxyribonuleotide acid monomer after precipitation and subsequent drying of the precipitate at 50° C. or lyophilisation of the DNA molecule at 50° C. in the presence of magnesium chloride
adding T4-DNA-ligase and
incubation for at least 10 minutes at 37° C. preferably for at least 30 minutes.

The same results with regard to the manufacture of a polymer can be obtained with the precipitation or lyophilisation in the presence of magnesium chloride, especially if the deoxyribonucleotide acid has been purified with a polyacrylamide gel electrophoresis, or with a combination of HPLC and FPLC.

It was completely surprising, that application of the method results in different molecular structures than the dimers, described in the state of the art (WO 2007/131495 or WO 01/07055. As the methods show only differences in several steps the more surprising is was, that the relatively slight modifications resulted in the manufacture of different molecules. Structures obtained with the method known from the state of the art (WO 01/07055 or WO 2007/131495) show significant differences in their properties. The molecules differentiate clearly with regard to the immune modulatory effect, but also in other characteristics, like for instance side effects. Besides the different steps of the methods, the use of educts with the preferred sequences leads to the formation of a very specific reaction product with specific and outstanding properties. The use of sequences according to the invention together with the above mentioned method steps results in advantageous polymers, showing preferred properties with regard to the ones from the state of the art.

A polymer according to the invention comprises preferably 2+2 monomers (comp. FIG. 1), preferred partly single stranded, covalently closed chains of deoxyribonucleotide components, wherein the monomers have a stem and a loop, wherein the stem has at least 2 deoxyribonucleotides and the loop at least 4 deoxyribonucleotides and the loop has 1 to 6 CG-motives and the variable n is an element from the set of all natural numbers.

The invention relates further to a composition, which comprises at least a molecule according to the invention and a chemotherapeutic. It was surprising that the unexpected strong improvement of the immune response by a molecule according to the invention could be further clearly improved by combining the remedy according to the invention with known chemotherapeutics and using the composition preferably for instance for the treatment of tumours. Although it was known by a person skilled in the art, that dimers according to WO 01/07055 have an immune modulatory effect and it was further known that chemotherapeutics have an effect on tumours, it was surprising that the immune modulatory dimers composed of monomers cause in combination with chemotherapeutics an over-additive effect. Still more surprising was that the polymers composed of monomers respectively the concatemers in combination with chemotherapeutics show a more positive effect than the dimers. The elements combined in a composition according to the invention have an effect on the same aim to treat pathogens, especially tumours. Each element does not define an isolated result within the composition according to the invention, but the interaction between the single elements leads to the surprising effect, which is more pronounced in the polymers than in the dimers. A composition according to the invention may be provided as a kit, in which a molecule according to the invention and the chemotherapeutics according to the state of the art are provided separately. Thus, in a preferred embodiment the at least two components of the kits may be applied simultaneously or time delayed. The application of a composition according to the invention may for instance activate the immune system so that a subsequent application of a chemotherapeutic may have a very good effect. It is a matter of course, that it is possible to apply at first the chemotherapeutic and subsequently with a time delay a molecule according to the invention into the human or animal organism. For defined tumours the simultaneous application of a molecule according to the invention and the chemotherapeutic is preferred.

In a preferred embodiment of the invention a chemotherapeutic is selected from the group comprising antibodies, alkylating agents, platinum analoga, intercalating agents, antibiotics, mitosis suppresses, taxanes, topoisomerases suppressors, anti-metabolites and/or L-asparaginase, hydroxycarbamide, mitotanes and/or amanitines.

In a preferred embodiment of the invention the alkylating agents are selected from the group comprising
nitrogen mustard derivatives, especially
cyclophosphamide,
ifosfamide,
trofosfamide,
melphalan and/or
chlorambucil
alkylsulfonate, especially
busulfan, and/or
treosulfan
nitrosourea, especially
carmustine,
lomustine,
nimustine
estramustine and/or
streptozotocin
procarbazine and dacarbazine,
temozolomide and/or
thiotepa.

The alkylating agents have a very good effect on tumours, inhibiting their growth.

In a preferred embodiment of the invention the platinum analoga are selected from a group comprising:
cisplatin,
carboplatin and/or
oxaliplatin.

In a further preferred embodiment of the invention it is intended, that the intercalating agents are selected from the group comprising:
anthracycline, especially
doxorubicine (adriamycin),
daunorubicine,
epirubicine and/or
idarubicine,
mitoxantron,
amsacrine and/or
doxifluridine.

In a further preferred embodiment of the invention it is intended, that the antibiotics are selected from the group comprising:
bleomycine,
actinomycine D (dactinomycine) and/or
mitomycine.

It can be furthermore intended in another preferred embodiment of the invention as an advantage, that the mitoses suppressers are to selected form the group comprising:
alkaloids of vinca rosea, especially
vinorelbine,
vincristine (oncovine),
vinblastine and/or
vindesine.

In a further especially preferred embodiment of the invention the taxanes are selected from the group comprising:
paclitaxel and/or
docetaxel.

Further it can be preferred, that the toposimerase suppressors are selected from the group comprising:
topoisomerase-I-inhibitors, especially
   camptothecin,
   topotecan and/or
   irinotecan and/or
topoisomerase-II-inhibitors, especially,
   etoposide,
   teniposide.

Further it is preferred that in a special embodiment of the invention the anit-metabolites are selected from the group comprising:
folic acid antagonist, especially
   methotrexat,
pyrimidin analoga, especially
   5-flouridacil,
   capecitabin,
   cytosine arabinoside (cytarabin) and/or
   gemcitabin,
purin analoga, especially
   6-thiogunaine,
   pentostatine,
   azathioprine,
   6-mercaptopurine,
   fludarabin and/or
   cladribine.

The invention relates further to a kit, comprising the molecule according to the invention and the chemotherapeutic, if applicable together with information about the combination of the content of the kit. The invention relates also—as already described—to a pharmaceutical comprising the molecule according to the invention or the composition if applicable with a pharmaceutical compatible carrier.

The invention relates further to the use of the molecule, the composition or the pharmaceutical for the manufacture of a remedy for the modulation of a human or animal immune system or for the modulation of the activity of the mentioned immune system. Modulation of the human or animal immune system shall be understood as each influence on the immune system, having the effect that the immune system inhibits tumours or cancer. The modulation of the activity of the immune system can synonymously be understood to this or be described for a person skilled in the art as the known activities of the immune system that are directed against tumours and being surprisingly increased in their effect by remedies according to the invention. The modulation is especially a stimulation or an increase of effects of the immune system respectively the immune system itself meaning a tumour-suppressive or remitting prophylactic effect. Thus a remedy according to the invention can be used in a preferred embodiment to stimulate the T-cell mediated immune response but also to change a T-cell independent immune response. This process may comprise in a preferred embodiment of the invention a proliferation of B-cells or B-cell activation.

In an especially preferred embodiment the modulation of the activity of the immune system results in an improvement with the effect that the secretion of cytokines of different relevant cell populations is changed respectively reverted. It may be especially preferred that the molecule according to the invention respectively the composition according to the invention are used as adjuvant in therapeutic or prophylactic vaccination. The remedy according to the invention may be used very efficiently for the treatment of cell growth disorders, wherein in a preferred embodiment the cell growth disorder is a tumour disease. Preferably the tumour disease is a disease selected from the group comprising tumours of the ear-nose-throat region, comprising tumors of the inner nose, nasal sinus, nasopharynx, lips, oral cavity, oropharynx, larynx, hypopharynx, ear, salivary glands, and paragangliomas, tumors of the lungs comprising non-parvicellular bronchial carcinomas, parvicel-lular bronchial carcinomas, tumors of the mediastinum, tumors of the gastrointestinal tract, comprising tumors of the esophagus, stomach, pancreas, liver, gallbladder and biliary tract, small intestine, colon and rectal carcinomas and anal carcinomas, urogenital tumors comprising tumors of the kidneys, ureter, bladder, prostate gland, urethra, penis and testicles, gynecological tumors comprising tumors of the cervix, vagina, vulva, uterine cancer, malignant trophoblast disease, ovarial carcinoma, tumors of the uterine tube (Tuba Faloppii), tumors of the abdominal cavity, mammary carcinomas, tumors of the endo-crine organs, comprising tumors of the thyroid, parathyroid, adrenal cortex, endocrine pancreas tumors, carcinoid tumors and carcinoid syndrome, multiple endocrine neoplasias, bone and soft-tissue sarcomas, mesotheliomas, skin tumors, melanomas comprising cutaneous and intraocu-lar melanomas, tumors of the central nervous system, tumors during infancy, comprising retinoblastoma, Wilms tumor, neurofibromatosis, neuroblastoma, Ewing sarcoma tumor family, rhabdomyosarcoma, lymphomas comprising non-Hodgkin lymphomas, cutaneous T cell lymphomas, primary lymphomas of the central nervous system, morbus Hodgkin, leukemias comprising acute leukemias, chronic myeloid and lymphatic leukemias, plasma cell neoplasms, myelodysplasia syndromes, paraneoplastic syndromes, metastases with unknown primary tumor (CUP syndrome) , metastasizing tumours comprising brain metastases, lung metastases, liver metastases, bone metastases, pleural and pericardial metastases and malignant ascites, peritoneal carcinomatosis, immunosuppression-related malignancy comprising AIDS-related malignancy such as Kaposi sarcoma, AIDS-associated lymphomas, AIDS-associated lymphomas of the central nervous system, AIDS-associated morbus Hodgkin and AIDS-associated anogenital tumors, transplantation-related malignancy.

In the following the invention is illustrated by examples without being limited to those examples.

Examples for the manufacture of the immune modulatory nucleic acid molecules:
a) Manufacture of the not claimed dimeric monomer:
5′-phosphorylated oligodeoxyribonucleotide (ODN) with the sequence CCTAGGGGTT ACCACCTTCATTGGAAAACGTTCTTCGGGGCGTTCTTAGGTGGTAACC (SEQ ID No. 3) were heated for 5 min to 90° C. and subsequently cooled on ice, to enable development of a hairpin structure. Self-complementary overhangs were ligated with a final concentration of 1 mg/ml DNA in the presence of T4-DNA Ligase (0,1 U/μg ODN) for 24 h at 37° C.
Separation of the purified ligation product on a 3% agarose gel, compare FIG. 2 lane 2.
b) Manufacture of a tetramer as example for the claimed polymers:
The degree of polymeristaion can only be influenced to a certain degree by the concentration of the employed nucleic acid. For the specific manufacture of a dimeric concatemer as shown in FIG. 2 the method of manufacture was modified as follows:
   5′-CCCTAGGGGTTACCACCTTCATTGGAAAACGTTCTTCGGGGCGTTCTTTCCCCAATGGTGGA-3′ (SEQ ID No. 4) and 5′-CCCTTCCACCATTGGGGATCATTGG AAAACGTTCTTCGGGGCGTTCTTAGGTGGTAACCCCT-3′ (SEQ ID No. 5) with equimolar concentrations (50 μM) were denatured for 5 min at 95° C. and subsequently slowly cooled for 50 min at 25° C.
   to this the 5′-phosphorylated nucleic acid with the sequence 5′-AGGGGTTACCACCTTCATTGGAAAACGTTCTTCGGGGCGTTCTTAGGTGGTAAC-3′ (SEQ ID No. 6) was added in a single molar excess
   all further steps were performed according to the above described method.
Separation of the pirified ligation product on a 3% agarose gel, compare FIG. 2 lane 2.
c) Manufacture of high molecular polymers:
Nucleic acids with the sequence 5′-pCTAGGGGTTACCACCTACAAAAAAA AACGAAATTCGGGGCGAAGGGAGGTGGTAACCC-3′ (SEQ ID No. 7) with a concentration of 1 mg/ml was precipitated with 0.3M sodium-acetate (pH 5,25), 1 OmM MgCl₂ and a threefold volume of ethanol abs. After centrifugation (4° C., 13000 rpm) the ODN was dried at 50° C. for 10 min. The pellet was directly used for ligation (0.5 U/μg ODN) and incubated for 60 min at 37° C. Separation of the ligation product on a 3% agarose gel, compare FIG. 2, lane 4.

### Description of FIG. 2:

In order to determine the molecular weights the manufactured molecules were separated on a3% agarose gel. The left lane 1 shows the molecular weights of double stranded DNA indicating the mass of each band, corresponding to the different migration distances. Lanes 2 to 4 were loaded with products of the different polymerisation reactions. A single band can be observed corresponding to the dimmer (lane 2) respectively the tetramer (lane 3) respectively to a ladder comprising all forms of polymers (lane 4).

Functional demonstration of molecules according to the invention:

Different cell culture experiments were done in order to prove the immune modulatory properties of the molecules according to the invention. The ability to stimulate TLR9 was investigated by use of murine macrophages of the cell line RAW 264 in which the expression of the Green Fluorescent Protein EGFP is under control of the positively by TLR9 regulated NF-κB promotor. The cells were seeded with 125000 cells/cm² and after 16 h the dimeric (manufactured according to method a) and polymeric (manufactured according to method c) molecules according to the invention were applied. After 7 h of incubation (37° C., 5% CO2) the cells were harvested and measured by EGFP expression was determined using fluorescence activated cell sorting (FACS). The results were used to generate a concentration-effect-curve, shown in FIG. 3; as molecular weight for both groups of molecules the dimeric weight was used as a basis in order to allow direct comparability.

The potency of the polymeric molecules according to the invention is increased by a factor of 10 (upper curve with closed symbols) in comparison to the low molecular weight molecules (lower dashed curve with open symbols). High molecular polymers according to the invention have a clearly better effect with equivalent amounts used as comparable amounts of dimeric or monomeric molecules. The higher potency for TLR9 stimulation can be attributed to a locally higher concentration achieved by the multimeric molecules which can especially in vivo not be achieved by higher doses, e.g. for reasons of the applicable amount. Simultaneously the high molecular concatemers have an increased efficiency which is completely surprising and can not be explained according to the current knowledge of sciences.

Stimulation of PBMCs for cytokine production

In order to perform stimulation assays peripheral mononuclear blood cells (PBMC) were isolated from whole human blood or so-called “buffy coat”. The isolated cells (PBMC) were seeded in multi-well-plates. The first mixture contained not stimulated cells as negative control, the second mixture was stimulated with dimers as comparison to the state of the art, the third with tetrameric polymers; the same mass of dimmers respectively polymers was used in the same volume. ELISA determined the secretion of the cytokines interferon-γ, interferon-α and interleukin-6 from the cell culture supernatant two days later, compare FIG. 4.

According to FIG. 4 the stimulation of PBMCs with the polymeric molecules according to the invention results in a doubling of the interferon secretion in comparison to the stimulation by dimeric molecules. The figure shows further that the IL-6 secretion due to stimulation with polymeric molecules is significantly higher compared with the stimulation with dimers.

It is possible to manufacture molecules according to the invention by using monomers with the following sequences:

a)

(SEQ ID No. 8)
5′-CTAGGGGTTACCACCTTCTATAGAAAACGTTCTTCGGGGCGTTCTT
CATCGGTAACCC-3′
or

b)

(SEQ ID No. 9)
5′-AGCTGGGTTACCACCTTCATTGGAAAACGTTCTTCGGGGCGTTCTT
AGGTGGTAACCC-3′
or

c) every 5′-terminally phosphorylated nucleic acid or mixture of nucleic acids with sequences being able to adopt a conformation as shown in FIG. 1, fulfilling the depicted conformation criteria and to hybridise with each other via single stranded overhangs (sticky ends) of at least four suitable nucleotide.
The deoxyribonucleic acid sequences used as educts are not heated prior to ligation and have a purification grade comparable to polyacrylamide electrophoresis. The educts can be purified by HPLC followed by FPLC. The combination of HPLC and FPLC results in an equivalent purification grade to polyacrylamide electrophoresis. Subsequently the DNA-educts are lyophilised at 50° C. until a dry residue is obtained. A resuspension in a buffer is made and T4-DNA ligase is added followed by an incubation at 37° C. for 40 minutes. It was surprising, that the obtained concatemers cause an improved immune modulation in mice. Surprisingly the combination of the single components of the concatenates according to the invention with chemotherapeutics results in an improved effect. The improved effect is surprisingly higher then the one of the single components and is beyond an additive effect. As chemotherapeutic antibodies, alkylating agents, platinum analoga, intercalating agents, antibiotics, mitosis suppresses, taxanes, topoisomerases suppressors, anti-metabolites and/or L-asparaginase, hydroxycarbamide, mitotanes and/or amanitines may be used.

1. A concatemeric molecule for the modulation of the activity of the human or animal immune system, wherein the concatemeric molecule comprises at least four deoxyribonucleic acid sequences as monomer units, which are covalently bound and comply with the formula:
B-M-{[Jᵢ-UᵢAᵢ]_{0 . . . n}}ₙ-E-K-T-{[Rₙ₋ᵢ₊₁Yₙ₋ᵢ₊₁1ₙ₋ᵢ₊₁]_{0 . . . n}}ₙ-G ∀ n ε IN₀, wherein
A, B, E, G, I, J, K, M, R, T, U, Y are deoxyribonucleotide molecules and
“—” represents a phosphordiester bond by which the nucleic acids are covalently bound to each other and
the sequence of component i of a nucleic acid molecule of the (i+1) of the same molecule may be different or not and
at least one nucleic acid comprises a motive with the deoxyribonucleotide sequence CG and
B, Uᵢ, K and Yₙ₋ᵢ₊₁ are predominantly single stranded and
B, Uᵢ, K and Yₙ₋ᵢ₊₁ are each assembled of at least 4 deoxyribonucleotides and
the sequences of Jᵢ to 1ₙ₋ᵢ₊₁, Aᵢ to Rₙ₋ᵢ₊₁, M to G respectively E to T are reveres complementary to each other and
G is covalently linked via a phosphodiester bound to B.

2. A molecule aacording to the previous claim, characterized in that the deoxyribonucleic acid used in the method comprises the following sequence:
(SEQ ID No. 1)
5′-GGGTTACCACCTTCATTGGAAAACGTTCTTCGGGGCGTTCTTA-
GGTGGTAACCC-3′
or
(SEQ ID No. 4)
5′-CCCTAGGGGTTACCACCTTCATTGGAAAACGTTCTTCGGGGC-
GTTCTTTCCCCAATGGTGGA-3′
or
(SEQ ID No. 5)
5′-CCCTTCCACCATTGGGGATCATTGGAAAACGTTCTTCGGGGC-
GTTCTTAGGTGGTAACCCCT-3′
or
(SEQ ID No. 6)
5′-AGGGGTTACCACCTTCATTGGAAAACGTTCTTCGGGGCGTTCT-
TAGGTGGTAAC-3′,
wherein the deoxyribonuclei acid has a length from 20 to 400 nucleotides.

3. Composition comprising a molecule according to claim 1 or 2 and a chemotherapeutic selected from the group comprising antibodies, alkylating agents, platinum analoga, intercalating agents, antibiotics, mitosis suppresses, taxanes, topoisomerases suppressors, anti-metabolites and/or L-asparaginase, hydroxycarbamide, mitotanes and/or amanitines.

4. Composition according to the previous claim characterized in that the alkylating agent is selected from the group comprising:
nitrogen mustard derivatives, especially
cyclophosphamide,
ifosfamide,
trofosfamide,
melphalan and/or
chlorambucil
alkylsulfonate, especially
busulfan, and/or
treosulfan
nitrosourea, especially
carmustine,
lomustine,
nimustine
estramustine and/or
streptozotocin
procarbazine and dacarbazine,
temozolomide and/or
thiotepa.

5. Composition according to one of the preceding claims, characterized in that the platinum analoga are selected from a group comprising:
cisplatin,
carboplatin and/or
oxaliplatin.

6. Composition according to one of the preceding claims, characterized in that the intercalating agents are selected from the group comprising:
anthracycline, especially
doxorubicine (adriamycin),
daunorubicine,
epirubicine and/or
idarubicine,
mitoxantron,
amsacrine and/or
doxifluridine.

7. Composition according to one of the preceding claims, characterized in that the antibiotics are selected from the group comprising:
bleomycine,
actinomycine D (dactinomycine) and/or
mitomycine.

8. Composition according to one of the preceding claims, characterized in that the mitoses suppressers are selected form the group comprising:
alkaloids of vinca rosea, especially,
vinorelbine,
vincristine (oncovine),
vinblastine and/or
vindesine.

9. Composition according to one of the preceding claims, characterized in that the taxanes are selected from the group comprising:
paclitaxel and/or
docetaxel.

10. Composition according to one of the preceding claims, characterized in that the toposimerase suppressors are selected from the group comprising:
topoisomerase-I-inhibitors, especially
camptothecin,
topotecan and/or
irinotecan and/or
topoisomerase-II-inhibitors, especially,
etoposide,
teniposide.

11. Composition according to one of the preceding claims, characterized in that the anitmetabolites are selected from the group comprising:
folic acid antagonist, especially
methotrexat,
pyrimidin analoga, especially
5-flouridacil,
capecitabin,
cytosine arabinoside (cytarabin) and/or
gemcitabin,
purin analoga, especially
6-thiogunaine,
pentostatine,
azathioprine,
6-mercaptopurine,
fludarabin and/or
cladribine.

12. Kit comprising a molecule according to one of the claim 1 or 2 and/or a composition according to one of the claims 3 to 11 and if applicable an information about combining the content of the kit.

13. Molecule according to one of the claims 1 to 2, composition according to one of the claims 3 to 11 for the use as medicament.

14. Pharmaceutical comprising a molecule according to one of the claim 1 or 2 and/or a composition according to one of the 3 to 11 if applicable together with a pharmaceutical compatible carrier.

15. Pharmaceutical according to the preceding claim, characterized in that the carrier is selected from the group comprising antibodies, alkylating agents, platinum analoga, intercalating agents, antibiotics, mitosis suppresses, taxanes, topoisomerases suppressors, anti-metabolites and/or L-asparaginase, hydroxycarbamide, mitotanes and/or amanitines.

16. Use of the molecule according to claim 1 or 2, the composition according to claims 3 to 11 or the pharmaceutical according to claim 14 or 15, for the manufacture of a remedy for the modulation of a human or animal immune system or for the modulation of the activity of the mentioned immune system.

17. Use according to the preceding claim, characterized in that the modulation is an increase of the activity of the immune system, wherein the activity of single cells or cell-subpopulations of the immune system is stimulated or accelerated or inhibited or attenuated.

18. Use according to the preceding claim, characterized in that the modulation comprises a T-cell mediated or -independent immune response.

19. Use according to the preceding claim, characterized in that the immune response comprises a proliferation of B-cells and/or a B-cell activation.

20. Use according to one of the preceding claim, characterized in that the stimulation of the immune system comprises a secretion of cytokines.

21. Use according to one of the preceding claim, characterized in that the molecule according to on of claim 1 or 2 and/or the composition according to claims 3 to 11 is used as adjuvant in therapeutically or prophylactic vaccination.

22. Use of a molecule according to claim 1 or 2, the composition according to claims 3 to 11 or the pharmaceutical according to claim 14 or 15, for the manufacture of a remedy for the treatment of cell growth disorders.

23. Use according to the preceding claim, characterized in that the cell growth disorder is a tumour disease.

24. Use according to the preceding claim, characterized in that the tumour disease is a disease selected from the group comprising tumours of the ear-nose-throat region, comprising tumors of the inner nose, nasal sinus, nasopharynx, lips, oral cavity, oropharynx, larynx, hypopharynx, ear, salivary glands, and paragangliomas, tumors of the lungs comprising non-parvicellular bronchial carcinomas, parvicel-lular bronchial carcinomas, tumors of the mediastinum, tumors of the gastrointestinal tract, comprising tumors of the esophagus, stomach, pancreas, liver, gallbladder and biliary tract, small intestine, colon and rectal carcinomas and anal carcinomas, urogenital tumors comprising tumors of the kidneys, ureter, bladder, prostate gland, urethra, penis and testicles, gynecological tumors comprising tumors of the cervix, vagina, vulva, uterine cancer, malignant trophoblast disease, ovarial carcinoma, tumors of the uterine tube (Tuba Faloppii), tumors of the abdominal cavity, mammary carcinomas, tumors of the endo-crine organs, comprising tumors of the thyroid, parathyroid, adrenal cortex, endocrine pancreas tumors, carcinoid tumors and carcinoid syndrome, multiple endo-crine neoplasias, bone and soft-tissue sarcomas, mesotheliomas, skin tumors, melanomas comprising cutaneous and intraocu-lar melanomas, tumors of the central nervous system, tumors during infancy, comprising retinoblastoma, Wilms tumor, neurofibromatosis, neuroblastoma, Ewing sarcoma tumor family, rhabdomyosarcoma, lymphomas comprising non-Hodgkin lymphomas, cutaneous T cell lymphomas, primary lymphomas of the central nervous system, morbus Hodgkin, leukemias comprising acute leukemias, chronic myeloid and lymphatic leukemias, plasma cell neoplasms, myelodysplasia syndromes, paraneoplastic syndromes, metastases with unknown primary tumor (CUP syndrome) , peritoneal carcinomatosis, immunosuppression-related malignancy comprising AIDS-related malignancy such as Kaposi sarcoma, AIDS-associated lymphomas, AIDS-associated lymphomas of the central nervous system, AIDS-associated morbus Hodgkin and AIDS-associated anogenital tumors, transplantation-related malignancy, metastasized tumors comprising brain metastases, lung metastases, liver metastases, bone metastases, pleural and pericardial metastases, and malignant ascites.
